(19) United States
(12) Patent Application Publication
(10) Pub. No.: US 2025/0226110 A1
(43) Pub. Date: Jul. 10, 2025

(54) SYSTEMS AND METHODS FOR FIELD SOBRIETY TEST DIGITIZING AND ANALYZING

(71) Applicants: Sean Muir, Corvalis, OR (US); Francisco Martin, Corvalis, OR (US)

(72) Inventors: Sean Muir, Corvalis, OR (US); Francisco Martin, Corvalis, OR (US)

(21) Appl. No.: 19/014,138

(22) Filed: Jan. 8, 2025

Related U.S. Application Data
(60) Provisional application No. 63/618,887, filed on Jan. 8, 2024.

(51) Int. Cl. G16H 50/30; G06T 7/20; G16H 30/20

(52) U.S. Cl. CPC G16H 50/30; G06T 7/20; G16H 30/20

(57) ABSTRACT

Standard field sobriety tests (SFST) are administered by a computing system that includes hardware and software configured to track body movement by receiving motion data and tracking one or more body landmarks including head, eyes, pupils, hands, feet, center of mass, and others to determine SFST clues associated with impairment. Machine learning techniques are trained to determine the captured motion presents SFST clues and determine a confidence level that the subject is impaired. The system can also record and store data associated with the SFST for later analysis or playback.

### BACKGROUND

The National Highway Traffic Safety Administration (NHTSA) has validated a number of tests in which a law enforcement office can administer in the field to test for sobriety. Currently, there are three such tests that have been validated, and together, these are known as a standardized field sobriety test (SFST). Specifically, the three validated tests include horizontal gaze nystagmus (HGN), walk and turn (WaT), and one leg stand (OLS).

These tests are often administered after a law enforcement officer pulls over a vehicle and its driver for suspected impairment. The offer will routinely administer the SFST on the side of a road near the officer's vehicle and/or the driver's vehicle.

However, there are many factors that may influence the administration or interpretation of the results of the SFST. For example, the driver may experience anxiety or nervousness, may be experiencing a medical condition that affects their ability to perform the SFST, the officer may be distracted by other events, radio calls, other vehicle, and the like. Moreover, the SFST requires a subjective administration and interpretation of the results, which cause uncertainties in the test results, which can lead to inadmissibility or lack of credibility in a later court case.

There is thus a need for a system and methods that can analyze a driver's SFST results with objective measures. There is a further need for a system that is capable of providing the aforementioned benefits while removing induced error caused by humans or their environment. These and other benefits will become readily apparent from the disclosure that follows.

### SUMMARY OF EMBODIMENTS

According to some embodiments, an objective method for determining impairment includes the steps of instructing a subject to perform a standardized field sobriety test (SFST); receiving video data of the subject; determining one or more body landmarks of the subject; tracking the one or more body landmarks during performance of the SFST to generate motion data; determining a score associated with the performance of the SFST; associating the score with the motion data; and generating a likelihood that the subject is impaired.

The method may further include capturing video data by a mobile phone and may also include receiving audio data of the subject. The method may determine an audio score associated with the audio data and determining the score associated with the performance of the SFST is based, at least in part, on the audio score.

In some examples, tracking the one or more body landmarks includes generating a bounding box around each of the one or more body landmarks. A machine learning model may be executed to correlate the motion data with the score. The one or more body landmarks may include a pupil, foot, center of mass, head, feet, arms, or other body landmarks.

According to some embodiments, a method for determining impairment of a subject includes the steps of instructing the subject to perform one or more standard field sobriety test (SFST); receiving video data of a body motion of the subject performing the one or more SFST; determining one or more body landmarks viewable in the video data of the body motion; tracking the one or more body landmarks during an action; generating, based at least in part on the tracking the one or more body landmarks, motion data; determining a score associated with the motion data; associating the motion data with the score; and generating, based at least in part on the motion data and the score, a likelihood that the subject is impaired.

In some cases, receiving the video data includes capturing the video data by a mobile computing device. A machine learning model may be executed to correlate the motion data with the score. The machine learning model may be trained on training data associated with performance of one or more SFSTs. The machine learning model may also be trained on other data sets relevant to interpretation of test results, such as medical information or bodily dimensions of subjects taking the test. Other data sources that quantify the level of alcohol and other substances in the subject that may cause impairment during the test, such as the a breath, urine, or blood-based tests, may also be used to train and improve the machine learning model. The machine learning model may also be trained using the direct input and feedback from human experts in impairment recognition (for example, Drug Recognition Experts). The machine learning training may be completed prior to deployment into the field or it may happen through direct feedback from the above mentioned data sources while operating in the field.

The method may include receiving an audible response and performing natural language processing on the audible response. In some cases, the method includes receiving motion data from a wearable sensor. The subject may be instructed by presenting, through a speaker, audible instructions for performing the one or more SFST.

In some examples, generating a likelihood that the subject is impaired is performed by a machine learning model. The machine learning model may be iteratively trained on training data through supervised learning.

According to some embodiments, a system for determining impairment of a subject through objective measures includes a data acquisition unit configured to capture video data and audio data of a subject performing a standardized field sobriety test (SFST), wherein the video data includes visual inputs of the subject's body movement and the audio data includes auditory inputs of the spoken instructions and responses; a computing device operably coupled with the data acquisition unit, the computing device comprising: a processor configured to execute a machine learning model that analyzes the video data to identify and track a plurality of body landmarks of the subject during the SFST to generate motion data, wherein the body landmarks include at least a pupil of an eye, a foot, and a center of mass; a memory storing instructions which, when executed by the processor, cause the computing device to process the audio data using natural language processing to assess speech patterns; a correlation module configured to integrate the motion data and assessed speech patterns to determine a score indicative of the subject's performance on the SFST; a decision engine operatively configured to generate, based on the score, a likelihood that the subject is impaired using a supervised machine learning algorithm trained on historical SFST performance data; and an output module configured to present the generated likelihood of impairment and associated analysis in a human-readable format.

The data acquisition unit may include a mobile computing device with an integrated camera and microphone, the camera positioned to capture a front-facing view of the subject, and the microphone configured to capture ambient noise and subject speech, wherein the captured data is utilized to enhance the accuracy of the motion data and speech pattern assessment by employing real-time noise reduction algorithms. For instance, a mobile phone, a body cam, a wearable device, a dash cam, or some other suitable data acquisition unit may be used with the systems and methods described herein.

### BRIEF DESCRIPTION OF THE DRAWINGS

The accompanying drawings are part of the disclosure and are incorporated into the present specification. The drawings illustrate examples of embodiments of the disclosure and, in conjunction with the description and claims, serve to explain, at least in part, various principles, features, or aspects of the disclosure. Certain embodiments of the disclosure are described more fully below with reference to the accompanying drawings. However, various aspects of the disclosure may be implemented in many different forms and should not be construed as being limited to the implementations set forth herein. Like numbers refer to like, but not necessarily the same or identical, elements throughout.

FIG. 1 illustrates a sample system for administering and analyzing results from an SFST, in accordance with some embodiments.

FIG. 2 illustrates some results of a machine learning model applied to a horizontal gaze nystagmus test, in accordance with some embodiments.

FIG. 3 illustrates an example of a scatter plot presentation of a gaze nystagmus test, in accordance with some embodiments.

FIG. 4A illustrates a foot placement results in a WaT test, in accordance with some embodiments.

FIG. 4B illustrates balance information associated with a WaT test, in accordance with some embodiments.

FIG. 5A illustrates a computer implemented impairment test, in accordance with some embodiments.

FIG. 5B illustrates a pose analysis based on the impairment test, in accordance with some embodiments.

FIG. 6A illustrates a user interface for selecting body landmarks to analyze during an impairment test, in accordance with some embodiments.

FIG. 6B illustrates a graph and analysis of motion data associated with body landmarks during an impairment test, in accordance with some embodiments.

### DETAILED DESCRIPTION

According to some embodiments, a system is described that uses computer vision and machine learning to significantly reduce the subjectivity naturally associated with Standardized Field Sobriety Tests (SFST). A SFST is typically a test that law enforcement personnel administer to a driver who has been pulled over (a “subject”) for suspected impairment. An SFST may include one or more physiological tests, such as a horizontal gaze nystagmus (NSG) test. Nystagmus is an involuntary jerking or rapid movement of the eyes. This test evaluates a subject's eye movements and includes one or more of receiving verbal instructions from a test administrator and then tests for a lack of smooth pursuit, distinct and sustained nystagmus, nystagmus prior to forty-five degrees, and a vertical gaze nystagmus.

An SFST may further include one or more divided attention tests, such as a Walk and Turn (WaT) test, or a One Leg Stand (OLS) test. The divided attention tests may require the subject to concentrate on two or more things at the same time, such as answering questions while performing physical actions. Of course, while the following disclosure uses SFSTs as examples to demonstrate the systems and methods described, it should be apparent that other tests may be employed with the described systems, such as other standard and non-standard impairment tests. Such tests may further include balance tests, such as the Romberg Balance Test as an example, a finger to nose test, one leg stand, one leg hop, tipping the head backwards, counting the number of raised fingers, reciting the alphabet, counting forwards or backwards, among others.

An SFST may be preceded by one or more pre-test medical questions. Medical factors may impact the accuracy of the SFST and provide important information in analyzing the results of the SFST. For example, questions such as: Are you sick or injured, are you currently taking any medications, do you have diabetes, are you hypoglycemic, have you seen a doctor or dentist recently, do you have any speech problems, do you have any hearing problems, do you have any balance problems, have you had any past head injuries, and have you have any past physical injuries are just a few of the possible pre-SFST questions that may be asked.

According to some embodiments, the system includes a machine vision and machine learning features that can track eye movements, balance and foot placement, the ability to understand and follow instructions, among other factors. This may be performed, in large part, by a computer vision system that can track several body landmarks simultaneously, and in some cases, associate the motion of one or more body landmarks with a level of sobriety. As an example, a system may identify and track any number of body landmarks, such as 3, or 5, or 11, or 17, or 21, or 25, or 30, or more body landmarks. As the system tracks the position of the landmarks, which may be in two dimensions, or in three dimensions, the system can associate motion of the landmarks with the subject's ability to balance, focus, gaze, walk stand on one leg, understand verbal instructions, speak clearly, among other things. The system may analyze the subject's ability to perform certain tasks or tests and interpret the results of the subject's performance to determine a likelihood of impairment.

The motion capture may be performed by one or more cameras aimed generally at the participant, and the system may further include one or more microphones, which may be associated with the one or more cameras. For instance, a mobile telephone may include both a camera and a microphone and be configured to capture both audio and video. Other such devices are usable with the systems and methods described herein, which may include one or more mobile or mounted video capture device and/or audio capture devices. In some cases, one or more of the cameras are associated with a mobile computing device, such as, for example, a smartphone, a tablet, a laptop, a digital personal assistant, and a wearable device (e.g., watch, glasses, body cam, smart hat, bracelet, armband, mobile computing device, etc.). In some cases, a wearable device may include a sensor, such as an accelerometer, a vibration sensor, a motion sensor, or otherwise to provide motion data to the system. In some cases, a wearable device is placed on the subject to capture motion data that can be analyzed by the system to determine impairment. In some embodiments, the system tracks body marker position over time and generates motion plots. For instance, the body marker positions can be tracked during one or more of the SFST, such as the one leg stand, or the WaT test. Other sensors may be used in combination with any other sensor and may include, in addition to a video capture device, a pressure plate, an accelerometer, a motion sensor, and the like.

In some cases, a model may be generated based on the body landmarks. In some instances, the body landmarks may include one or more of a nose, left ear, left eye, left hip, left knee, right ear, right eye, right hip, left ankle, left elbow, left wrist, right knee, right ankle, right foot, left foot, right elbow, right wrist, left shoulder, right shoulder, head, among others. Of course, other body landmarks are contemplated. In some embodiments, a single camera may capture two-dimensional motion data associated with one or more of the body landmarks. In some examples, two or more cameras may be used to capture three-dimensional motion data of the one or more of the body landmarks.

The system may normalize the motion data to generate normalized coordinates of the position of each body part during all the SFSTs. The captured motion data and/or the audio data (collectively, “SFST data”) may be stored in a data file that can be analyzed in either near-real time and may also be saved for later analysis.

Normalization of data in the context of machine learning involves transforming input data into a format that enables more efficient and effective processing by a machine learning algorithm. This process often entails scaling the individual data features to a common scale without distorting differences in the ranges of values. For instance, when utilizing algorithms that rely on motion or distance calculations, such as K-Nearest Neighbors or Support Vector Machines, unnormalized data can lead to features with larger ranges disproportionately influencing the results. Common techniques for normalization include Min-Max scaling, which rescales the data to a specified range, typically between 0 and 1, and Z-score normalization, which scales data based on the standard deviation from the mean, centering the data around zero. By normalizing data, the model can perform better optimization, leading to faster convergence during the training process and improved overall accuracy and generalization. Additionally, normalization can assist in reducing redundancy and computational overhead, allowing the machine learning model to focus on learning inherent data patterns and relationships without bias imposed by variation in data magnitudes.

The motion data may be associated with SFST performance, such as the OLS, the WaT, or other such tests that require the subject to perform a motion. The audio data may likewise be stored for analysis, such as for speech patterns, which may include slurring, mispronouncing, inability to follow directions, and the like. The audio analysis may further be used to compare the speech patterns during the SFST with speech patterns at a time other than the SFST. According to some embodiments, a system is provided for administering the SFST, recording SFST, analyzing SFST recordings, and presenting the results of the SFST.

One or more machine learning approaches may be applied to the captured data and used to generate correlations between the SFST data and impairment. For example, in some embodiments, convolutional deep neural networks (DNN) may be designed to locate features in a collection of SFST data. Other deep-learning models that are oriented toward classification may also be used to correlate the SFST data to identify patterns that allow the system to determine impairment with a high level of confidence.

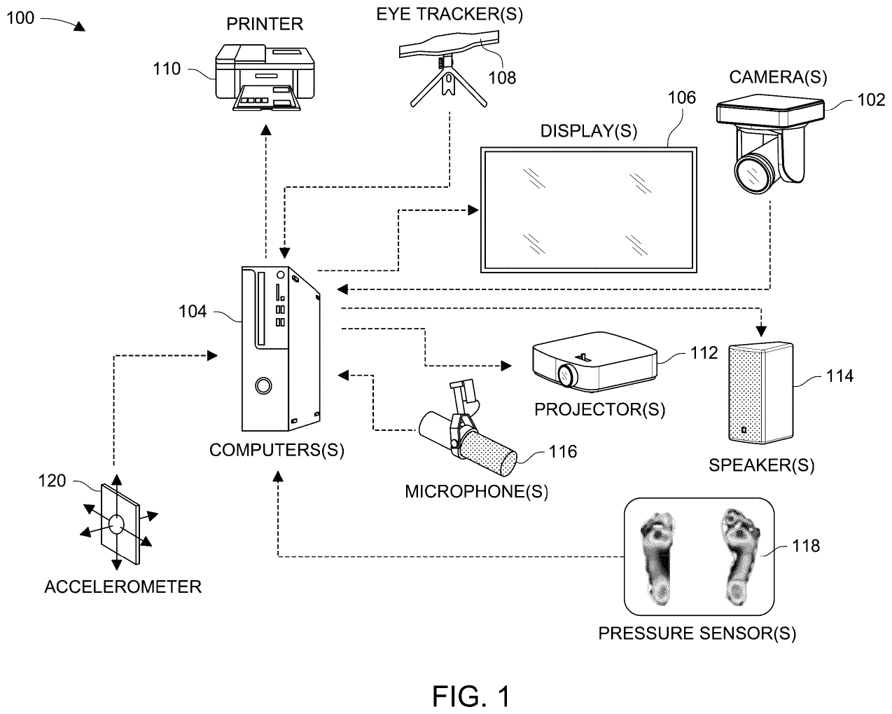

FIG. 1 illustrates and example embodiment of a system for administering and analyzing results from an SFST, in accordance with some embodiments. It should be appreciated that the components shown are not all necessary in some embodiments of the systems described herein. A system 100 may include one or more of a camera 102, a processor 104, a display 106, an eye tracker 108, a printer 110, a projector 112, a speaker 114, a microphone 116, a pressure sensor(s) 118, and an accelerometer(s) 120, which may be located in a wearable device.

According to some embodiments, one or more cameras 102 may be used to capture video data of a subject, such as when performing one or more SFSTs. The video data may be used by a machine learning algorithm, such as to determine a confidence level of impairment, which may be by intoxication or some other impairment. The video data may further be stored for later analysis, review, or other use. One or more computers 104, which may include a mobile computing device, desktop, laptop, tablet, remote computer, cloud-based computing devices, or other may have one or more processors configured with instructions that, when executed, cause the computer to carry out actions. Many of those actions are described herein and relate to capturing performance of a SFST and analyzing and/or outputting the results of the SFST. A display 106 may be implemented with the system 100 and may display results of an SFST test or may be used as part of the SFST, such as to display objects for the subject to track with their eyes or identify a pattern or shape, or present text for the subject to read.

An eye tracker 108 may be used with the system 100 to capture images of the eyes of a subject and may include eye motion of the subject, as described herein in conjunction with one or more of the SFSTs or for identification, or some other purpose. A printer 110 may be used such as for printing SFST results, or other documents.

In some cases, a projector 112 may be used for projecting an image, pattern, text, or otherwise and may be used in conjunction with an SFST, such as to display a line on the ground along which the subject is instructed to walk. One or more speakers 114 may be used to provide prompts to a subject, such as for providing instructions for performing one or more SFSTs or other field tests. A microphone 116 may be used to capture audio data associated with the SFST, or with a traffic stop in general, or for capturing audio data of an interaction with a subject. One or more pressure sensors 118 may be used, for instance, to generate data indicative of the balance of a subject and may include pressure sensors 118 configured to capture pressure data associated with a subject walking, turning, standing, standing on one leg, hopping, and the like. One or more accelerometers 120 may be used to capture motion data of a subject, such as while walking, standing, or performing the SFST. The accelerometer 120, may be carried by the subject or worn by the subject.

In some cases, one or more of the described components may reside in a single device. For instance, a mobile computing device, such as a smart phone or a tablet computer may each include a processor 104, a camera 102, a speaker 114, an eye tracker 108, a display 106, or other components described.

In some cases, the system is configured to use one or more cameras 102 for machine vision of a subject, determine one or more body landmarks (in some cases up to 17 or more body landmarks) and track the movement of each of these landmarks over time during a SFST. The system can correlate the time-bound body landmark movement with an impairment determination and determine a likelihood that the movement correlates with impairment. Furthermore, by looking at several SFSTs over time, the system can correlate subject behaviors with a level of impairment.

In some embodiments, the system utilizes a gantry type arrangement in which a plurality of recording devices (e.g., cameras and/or microphones) may be used to capture audio and video of the subject. In some cases, the system is configured to synchronize multiple sources, such as one or more video frames, and/or audio data from one or more audio capture devices. In some cases, the system is configured to synchronize multiple video frames and audio data from one or more audio/video capture devices.

The system may utilize one or more machine learning models for synchronization, analysis, verification, and may further be trained to analyze SFST data and associate the SFST data to determine correlations between specific motion data (e.g., behaviors) and impairment determinations. As an example, the system may correlate that a subject's balance during the OLS test is consistent with a person who is impaired. Therefore, when the system recognizes similar behavior in multiple subjects, it can determine with a reasonable degree of confidence that a lack of balance, or a particular combination of movements in an attempt to maintain balance, is related to impairment. A similar process may be used with any motion data from any SFST or other test, as may described elsewhere herein.

In some embodiments that utilize multiple video capture devices 102, the system may track body motion in two or three dimensions and from multiple angles. The two- or three-dimensional body motion data may be correlated, synchronized, and analyzed to determine two- or three-dimensional motion data, which can be further correlated with a resulting impairment score.

While embodiments of the described system are described in relation to a subject performing SFST, it should be understood that the systems and methods described herein are applicable to capturing any type of body motion and applicable to other sobriety tests where body motion may lead to performance. For example, embodiments of the systems described herein may be used to track, analyze critique, and determine an impairment score for activities such as, without limitation, touching one's nose, walking heel to toe, spinning in place, reciting a string of words or letters, answering questions, hopping up and down, standing on one's toes, among other motion or audio performance where the movements of a set of observable body landmarks or voice can be recorded over time and there is some observed causal consequence of the performance.

The system may include one or more processors and one or more computer readable media that may store various modules, applications, programs, or other data. The computer-readable media may include instructions that, when executed by the one or more processors, cause the processors to perform the operations described herein for the system.

In some implementations, the processor(s) may include a central processing unit (CPU), a graphical processing unit (GPU), both CPU and GPU, a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) may possess its own local memory, which also may store program modules, program data, and/or one or more operating systems. The one or more control systems, computer controller and remote control, may include one or more cores.

The computers 104 may execute one or more machine learning models to aid in determining whether a subject is impaired and/or quantify the level of impairment. The computers 104 may receive data from one or more sources, such as any of the devices described in relation to FIG. 1, and may further include additional data, such as prior subject data that may be compared with more recent subject data. The computer 104 may also be used to receive, record, analyze and interpret other relevant data, such as medical information, the results of other tests that quantify the level of alcohol and other substances in the subject, such as breath, urine, or blood-based tests. The computer 104 may also receive, record, analyze and interpret the direct input and feedback from human experts in impairment recognition (for example, Drug Recognition Experts).

Embodiments may be provided as a computer program product including a non-transitory machine-readable storage medium having stored thereon instructions (in compressed or uncompressed form) that may be used to program a computer (or other electronic device) to perform processes or methods described herein. The computer-readable media may include volatile and/or nonvolatile memory, removable and non-removable media implemented in any method or technology for storage of information, such as computer-readable instructions, data structures, program modules, or other data. The machine-readable storage medium may include, but is not limited to, hard drives, floppy diskettes, optical disks, CD-ROMs, DVDs, read-only memories (ROMs), random access memories (RAMs), EPROMs, EEPROMs, flash memory, magnetic or optical cards, solid-state memory devices, or other types of media/machine-readable medium suitable for storing electronic instructions. Further, embodiments may also be provided as a computer program product including a transitory machine-readable signal (in compressed or uncompressed form). Examples of machine-readable signals, whether modulated using a carrier or not, include, but are not limited to, signals that a computer system or machine hosting or running a computer program can be configured to access, including signals downloaded through the Internet or other networks.

In some embodiments the machine learning models include any suitable algorithm, such as deep neural networks, linear regression, random forest, decision trees, naïve bayes, and may include supervised or unsupervised learning, or a combination of training techniques. In some cases, the machine learning models undergo a training phase. In the case of supervised learning, a machine learning algorithm may learn from labeled examples. It may attempt to establish patterns or relationships between input data (features) and corresponding output labels. During the training phase, the algorithm adjusts its internal parameters iteratively to minimize the error or difference between its predicted output and the true labels in the training data.

In some cases, a deep neural network is composed of multiple layers of interconnected nodes called neurons. Each neuron applies a mathematical function to its inputs and passes the result to the next layer. The layers closer to the input are called the input layers, while those closer to the output are known as the output layers. Between them, there can be multiple hidden layers. These layers can correlate input data with output data based on patterns and training data.

In some cases activation functions introduce non-linearity to the neural network, allowing it to learn complex patterns. Common activation functions include sigmoid, ReLU (Rectified Linear Unit), and tanh (hyperbolic tangent). They may determine whether a neuron should be activated and to what extent.

In some embodiments forward propagation flows data through the neural network from the input layer to the output layer. At each layer, the inputs are multiplied by weights, passed through activation functions, and the results are sent as inputs to the next layer. This process continues until the output layer produces a predicted output.

In some examples, a loss function is used to measure the discrepancy between the predicted output and the true labels. It quantifies the error made by the algorithm during training. Common loss functions include mean squared error, cross-entropy, and softmax loss, depending on the type of problem being solved.

In some instances, backpropagation is a process used for training a deep neural network. It may involve calculating the gradients of the loss function with respect to the network's weights and biases. These gradients are used to update the weights and biases of the network in the opposite direction of the gradient, aiming to minimize the loss.

Finally, optimization algorithms, such as stochastic gradient descent (SGD) or its variants (e.g., Adam or RMSprop), may be used to update the network's parameters based on the calculated gradients. These algorithms adjust the weights and biases in a way that progressively reduces the loss, allowing the model to improve its predictions.

The training steps may be repeated iteratively for a defined number of epochs or until the model's performance reaches a satisfactory level. Each iteration enhances the model's ability to make accurate predictions by fine-tuning the internal parameters based on the training data.

After training, the machine learning algorithm may be evaluated using separate validation or test datasets. This may provide an unbiased measure of the algorithm's generalization capability. It allows for the detection of overfitting (when the model performs well on training data but poorly on new data), and helps in selecting the best-performing model.

In some embodiments, a trained machine learning algorithm is used for prediction or inference. It takes new, unseen data as input and applies the learned patterns to generate predictions or make decisions based on the problem it was trained for. The system 100 is thus configured to determine a likelihood of impairment of a subject based upon a variety of factors, including performance on SFST tasks.

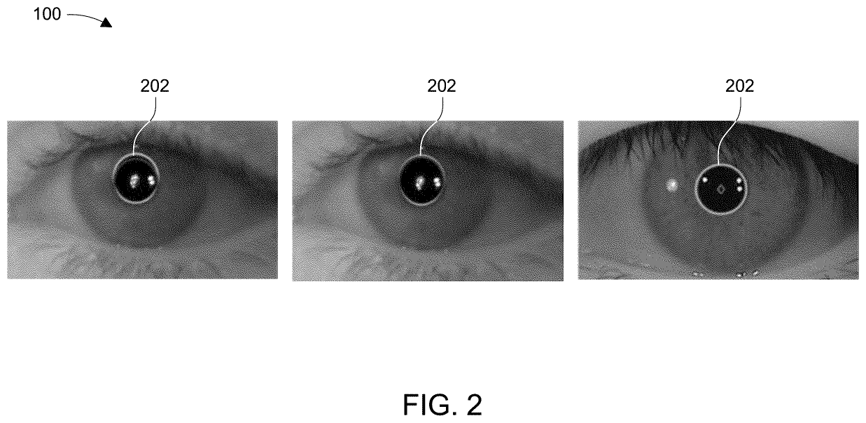

FIG. 2 illustrates results of a machine learning algorithm used to identify a subject's pupils, such as during an HGN test. The system 100 may be configured to track +pupil movement and/or head movement and may combine with other analyses and data inputs to determine the presence of SFST clues, test validity, or other factors that may affect the results of the HGN test. As illustrated, the system 100 may employ a machine learning algorithm that analyze a sequence of images and determine a location of the pupil 202. This may be done, for example, by any suitable image analysis algorithm, including without limitation, k-nearest neighbor, random forest, decision tree, support vector machine, naïve bayes, among others. By identifying the pupil, the system 100 can then compare the location of the pupil in subsequent frames of video data and determine pupil movement. In some cases, the pupil movement as a result of head movement may be isolated from pure pupil movement.

The result of the analysis may be a recording or animation of the eyes and may include markers or indicators added to highlight the presence of clues, or one or more scatter plots may be created to represent the movement of the eyes during the test and indicate the presence of SFST clues. As used herein, clues, or SFST clues refer to observable indicators of impairment and may include a variety of factors that are observable or discoverable during the administration of an SFST and may be used by the system 100 and/or law enforcement personnel as an indicator of impairment or level of impairment.

The system utilizes advanced computer vision techniques and machine learning algorithms to analyze the subject's eye movements as part of the assessment for identifying clues of impairment. By capturing video data of the subject's eyes during a horizontal gaze nystagmus test, the system can track specific eye characteristics such as smooth pursuit, involuntary jerking, and onset of nystagmus at certain angles. The system employs image processing techniques to detect and isolate the pupil and/or the iris within successive video frames, allowing for the precise measurement of eye movements. Through supervised machine learning, the system can be trained on a dataset containing eye movement patterns corresponding to various levels of sobriety and impairment. It compares the live data against this trained model to discern the presence of recognized impairment markers, such as lack of smooth pursuit or early onset of nystagmus. This automated evaluation reduces the reliance on subjective human interpretation, instead providing an objective analysis with higher precision and consistency across varying environmental conditions. The use of eye movement analysis as a biometric indicator significantly contributes to the system's ability to accurately determine the likelihood of impairment in a subject.

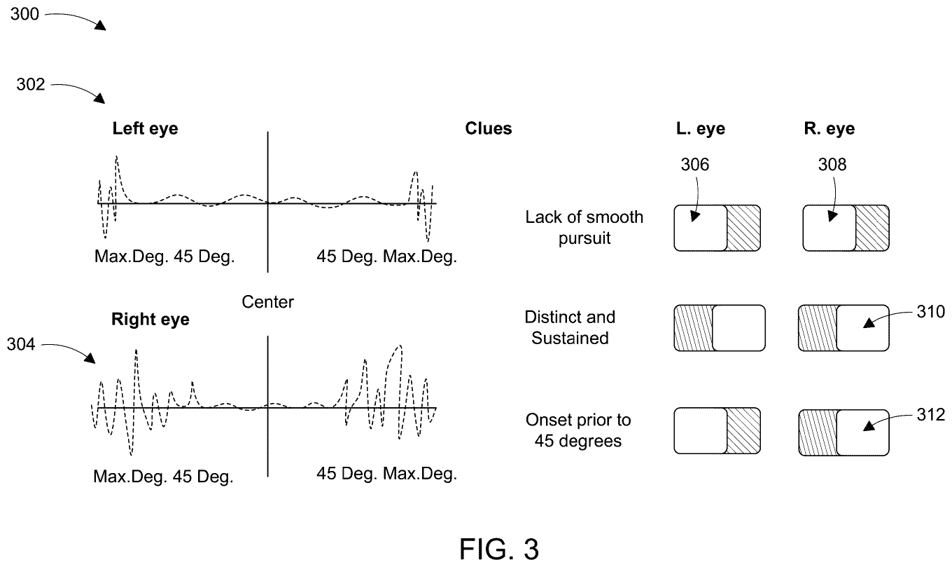

FIG. 3. Illustrates a possible scatter plot presentation of pupil movement following administration of an HGN test. Nystagmus is the medical term used to describe the involuntary jerking of the eyeballs. When someone is intoxicated by alcohol and/or certain drugs, this jerking becomes more pronounced. As such, the HGN test is used by law enforcement officers to evaluate a subject's nystagmus in order to determine if probable cause exists for a drunk-driving arrest. Prior to administering the HGN test, the officer may evaluate the subject's eyes to look for evidence of a resting nystagmus, equal pupil size and equal tracking (that is, if both eyes can follow an object together). If any of these factors are exhibited, there is chance of an existing medical condition or injury that may render the test results unreliable.

In administering the test, the officer will hold an object approximately 12-15 inches from the subject's nose and slowly move it from one side to the other. The officer instructs the subject to follow the object with their eyes while keeping their head still.

The officer will look for three different clues in each eye (for a total of six clues) during the test. One clue is to observe whether there is a lack of smooth pursuit, that is, the eyes jerking or bouncing while following the object from one side to the other. The object may typically be a tip of a pen, a fingertip, or a small light. Next, the officer will look for nystagmus that sets in before the eyes reach a 45-degree angle. A third clue is nystagmus at maximum deviation, or an observation that the eyes begin jerking within four seconds while looking all of the way to the side. If the officer observes four or more clues, they have probable cause to make an arrest for driving under the influence.

In some cases, it may be difficult for an officer to recognize or observe the various clues. For instance, lighting may be less than ideal, the officer may be distracted, or some other environmental factor may prevent the officer from properly administering and interpreting the results of the HGN test.

According to some embodiments, the pupil movements may be analyzed by one or more machine learning algorithms to identify patterns and clues that associate the movements with impairment. For example, the HGN test may test each eye which can yield clues for each eye individually. By using systems and methods described herein, the subjectivity associated with human-administered HGN tests is replaced by an objective machine vision system that is configured to track the eye movements of a subject and determine the presence of the four or more clues. In some cases, an observation of 4 or more clues typically indicates a 0.08% BAC or higher, with 88% accuracy.

In administering the HGN test, the system 100 may utilize a speaker which may be configured to play instructions to the subject, which may be able to play the instructions in multiple languages. The system 100 may further include a microphone that may be configured to receive audio input, such as subject responses to prompts or questions. A natural language processing module may be configured to receive the audio input from the subject's verbal responses and convert the audio input into machine readable form, text, or some other useful format.

A display may be used to display moving stimulus and may be configured to modify the stimulus shape, color, appearance, and the like. An eye tracking component may record eye movement and may rely on visible, infrared, near infrared, or some other spectrum of light in order to track the eye movement. In some cases, the eye tracking is performed by an eye tracker device, while in other cases, a camera is used to record the eye movement and a machine learning algorithm may be used to determine eye movement during the HGN test. The system may further include a method for analyzing the eye movement and determining whether the lack of smooth pursuit, distinct and sustained nystagmus at maximum deviation, or onset of nystagmus prior to 45 degree clues are present in either eye. The system may finally include a way to display the HGN analysis results. The display may include a visual display that shows text, a visual indicator that may include lights, symbols, a pattern, or an audible indicator that is associated with the HGN test results. The results of the HGN test may be stored locally, sent over a network, stored remotely, displayed on a screen or projector, printed out in a hard copy, or a combination.

One or more artificial intelligence, machine learning, or neural network models and processes may be employed to analyze the HGN test recording in order to identify and track the subject's pupil movement throughout the HGN test. The system may utilize currently existing technologies and models for eye, gaze, and head tracking and pupil detection, technologies and models customized for SFST clue detection, or other currently undeveloped technologies and model that enable eye, gaze, head tracking and pupil detection.

In some cases, a combination of pupil movement and head movement are combined to determine the presence of SFST clues, test validity, or other factors that may affect the results of the HGN test. Additional information may be associated with the test and stored in conjunction with the HGN test. The additional information may include one or more of the date and time of test administration, the identity of the subject, the identity of the HGN test administrator, the location of the HGN test, the current weather including temperature, humidity, precipitation, amount of natural light, among other factors. The data may further include a detailed list of test parameters including the type of stimulus, the length of the test, audio recordings associated with the HGN test, along with other information.

The result of the HGN test analysis may be a recording or animation of the eyes with markers and indicators added to highlight the presence of clues, or one or more scatter plots, such as shown in FIG. 3. The scatter plot may represent the movement of the eyes during the test and indicate the presence of SFST clues. For example, FIG. 3 indicates an example presentation 300 of HGN test results following analysis by the system described herein. The system may track movement of each eye during the test and may indicate movement of the left eye 302, movement of the right eye 304 with a plot showing the movement of each eye during the test. The display 300 may further indicate the presence of one or more clues associated with the eye movement. For instance, a lack of smooth pursuit of the left eye 306 and the right eye 308 may be indicated by a color changing indicia that indicates the presence or absence of the smooth pursuit clue. Similarly, indicia may be presented to show whether the subject was able to be distinct and sustained 310 during the test. Additionally, indicia may be presented that shows a clue related to onset prior to 45 degrees 312 of one or both eyes. Of course, the display of the test results and the clues displayed are only exemplary, and other tests, clues, and indicia may be used to illustrate the presence of one or more clues associated with eye movement during a test.

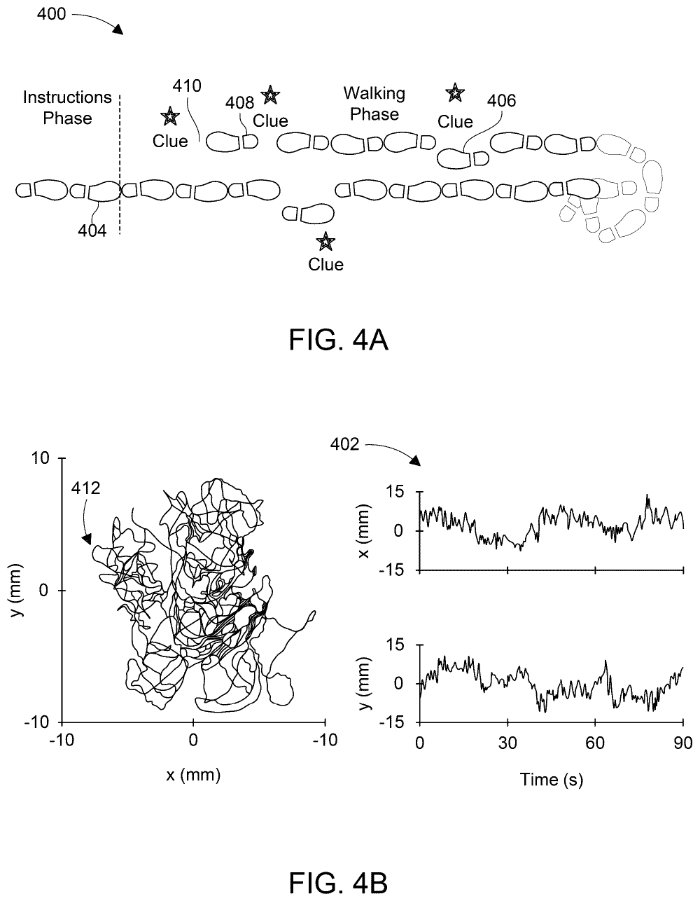

FIGS. 4A and 4B illustrate a walk and turn (WaT) test 400 and its results 402, respectively. The WaT test is designed to monitor the subject's ability to follow instructions, balance, and foot placement in response to the subject being asked to walk a straight line, turn around, and walk a straight line back according to a specific procedure that outlines how many steps each direction and how to turn around.

The walk and turn test includes an instruction stage and a walking stage, and each stage may provide clues. For example, during the instruction stage, if the subject fails to maintain balance or begins walking too soon, these are both clues that indicate impairment. The walking stage requires the subject to walk in a straight line a predetermined number of steps, such as 9 in some cases, perform a turn, and walk back to the starting point. The walking stage typically yields 6 distinct clues such as, for example, stopping while walking, misses touching the toe to the heel, steps offline, raises arms for balance, performs an improper turn, and performs the wrong number of steps. In some cases, where 2 or more clued are observed, the result is a BAC of 0.08% or greater with a 79% accuracy. The test may require some hardware and/or software components, which may include one or more of a way to convey instructions to the subject, which may be visual or audible; a microphone that can receive audible responses from the subject and a natural language processing module configured to interpret the subject's responses; a projector for displaying a straight line for the subject to walk which may include varying the width, length, color or ornamentation of the line; a way to record the instructions and procedure; components to determine the subject's balance and foot placement before, during and after following the described procedure; a system to analyze the WaT test performance to determine whether the subject fails to maintain balance, starts too soon, stops while walking, misses heel to toe, steps offline, raises arm for balance, performs an improper turn, or takes the wrong number of steps. The hardware may include a camera, a pressure sensor, a wearable device, or some other hardware configured to generate data associated with the performance of the WaT test.

The system may be configured to use artificial intelligence, machine learning, machine vision, and/or neural network models and processing for identifying and tracking a center of mass of the subject, movement of arms, hands, feet, and other factors related to detecting SFST clues as part of the WaT test analysis.

In some cases, one or more pressure sensors may be used in performance of the test and may alternatively or additionally be used in training the AI, M, or NN models to further improve their ability to detect clues. The results of the WaT test analysis may be stored locally, sent over a network and stored remotely, presented in visual format, presented in an audio format, or printed out or a combination.

FIG. 4A shows a possible visual output of the WaT test in which the subject's foot steps 404 are shown and deviations 406 from the instructions are highlighted or otherwise shows as SFST clues. For example, where the subject steps offline 406, missed heel to toe placement 408, or stops at a wrong location 410 are all indicative of SFST clues.

FIG. 4B illustrates a further visual output of the WaT test in which a scatter plot 412 may show the subject's center of mass, movement and placement of feet, and indicators of SFST clues.

Similar to the administration and output of the WaT test, a One Leg Stand (OLS) test may be administered that monitors the subject's ability to follow instructions and balance on one leg in response to the subject being asked to follow a specific procedure that outlines how to hold the foot and how long to keep it raised. The OLS test provides an opportunity for several clues to demonstrate impairment, such as identifying whether the subject sways, raises arms for balance, puts their foot down, or hops on one foot. These clues may be evaluated in discrete time periods, such as between 0 and 10 seconds, between 11-20 seconds, between 21-30 seconds or for some other period of time. In some cases, the system can be configured to indicate to the subject when to begin the test and it may automatically time the test and determine the SFST clues and the time period during which they manifest.

The OLS test may include hardware and software components, such as described herein with respect to any described embodiments and may include a way to convey instructions to the subject, perhaps in multiple languages; a device to respond to the subject either visually or audibly, a way to record the instruction and the procedure, a way to record the subject's balance and foot placements before, during, and after they follow the described procedure, a way to analyze the performance to determine whether the subject sways during test, raises arms, puts foot down, or hops, which each indicate an SFST clue. Finally, a way to present the test results and/or analysis of the test results may include visual, audible, or some other form of presentation of the results.

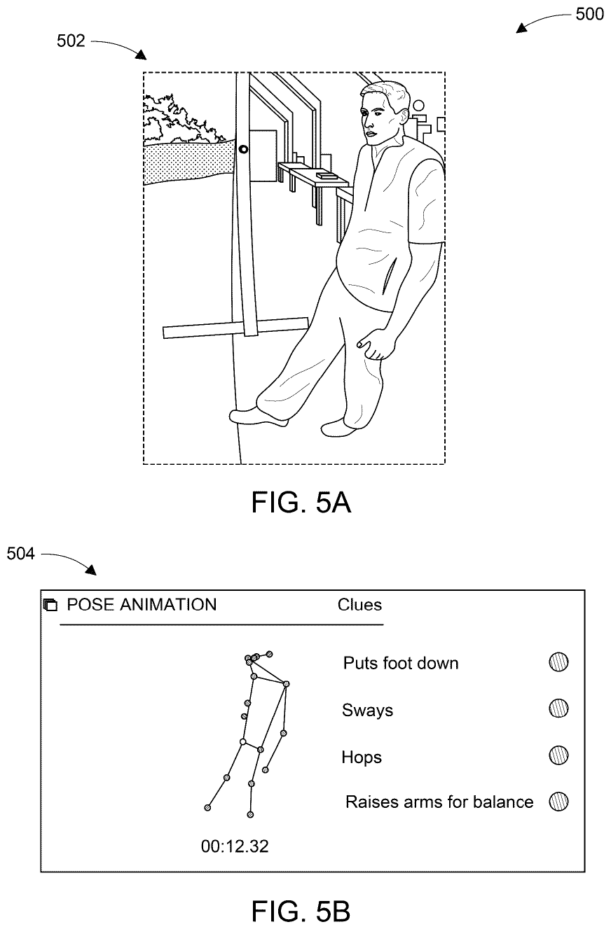

FIGS. 5A and 5B illustrate a one leg stand test 500. FIG. 5A shows video data of a test subject 502 captured by one or more cameras from one or more different angles. The cameras may be any suitable cameras, and may also be associated with a computing device, such as a mobile computing device. The one or more cameras may be associated with a smart phone, or other mobile computing device, or a mounted camera, such as a body camera, smart glasses, a vehicle mounted camera, a stationary mounted camera, or otherwise.

The one or more cameras may be pointed at the test subject and used to capture video and/or audio data of an impairment test. FIG. 5B illustrates a wireframe body model 504 generated by one or more computing devices that receive and analyze the video data. In some cases, such as is illustrated, the computing device uses one or more machine learning algorithms to detect body landmarks and tracks the motion of the body landmarks during the impairment test. The system may generate an animation of the wireframe body model based on the motion of the body landmarks. The wireframe body model may illustrate the motion of the subject while performing the SFST and be analyzed, along with the motion of the body landmarks, to identify clues of impairment. In some cases, the wireframe body model illustrates the relative positioning between the various body landmarks during motion of the subject. The motion of the body landmarks may indicate one or more markers of impairment. As an example, where the body landmark motion indicates that the test subject put a foot down, swayed, hops, or raises an arm for balance may be determined by analyzing the body landmark motion data, which may each be a marker of impairment.

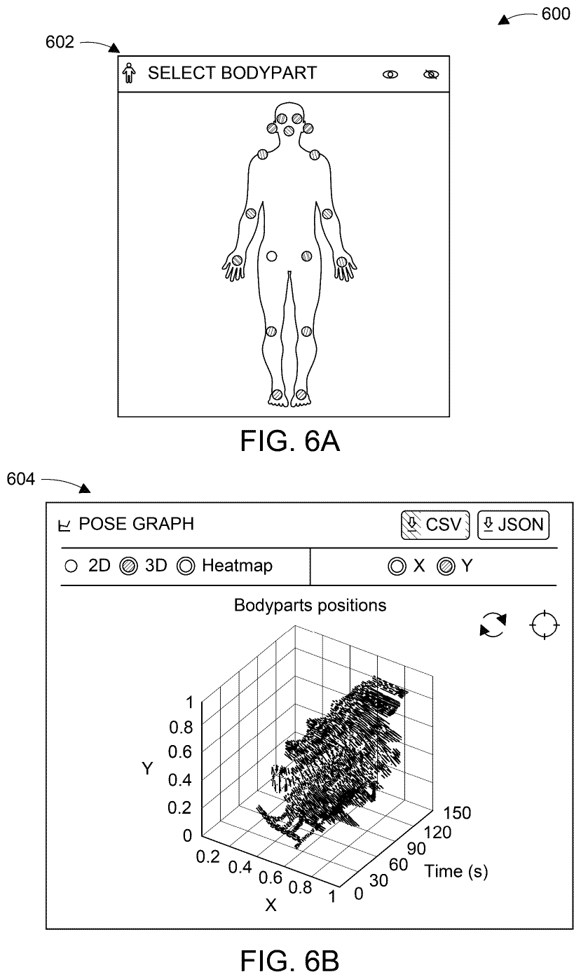

FIG. 6A illustrates an example user interface 600 of a computer program configured to track and analyze body landmark motion data. As an example, the user interface 600 may allow selection of one or more body landmarks 602 for tracking. In some examples, the computer program determines which body landmarks to select for tracking, while in some examples, a user may select one or more body landmarks for motion tracking. The body landmark selection may be based on which SFST is being performed. Of course, in some cases, the body landmark selection may be a combination of automatic selection and user selectable body landmarks.

FIG. 6B illustrates the motion data associated with the one or more body landmarks. The motion data may be captured as two-dimensional or three-dimensional motion data which can be displayed, played back, stored, or analyzed for impairment markers. The system may utilize any of a number of machine learning algorithms to determine a level of impairment, such as by detecting markers or clues of impairment based upon one or more impairment tests. The system may further count the number of times a clue is observed, the time at which the clue was observed and save the results for further analysis. The system may further output data associated with the impairment, such as data regarding the type and number of impairment clues, a probability that the test subject is impaired, and a confidence level of the impairment determination.

The system may include a computing device that may comprise artificial intelligence, machine learning, or neural network models and processing for identifying and tracking the location and/or motion of the subject's center of mass, arms, hands, feet, and other factors related to detecting SFST clues as part of the SFST analysis.

The systems and methods described herein leverage advanced computer vision and machine learning technologies to address the challenges associated with the subjective administration and interpretation of Standardized Field Sobriety Tests (SFSTs). Specifically, the invention employs a sophisticated arrangement of sensors and computing resources to capture, process, and analyze body movements and audio inputs to reduce the dependency on human interpretation, thereby yielding a more objective and reliable assessment of impairment.

The computing architecture integrates with multiple hardware peripherals, such as cameras, microphones, and optionally motion sensors, creating a multi-modal data acquisition system. This system is not merely a rehash of existing SFST methods but implements a novel technique of real-time data processing through neural networks designed for motion analysis and interpretation. The neural networks capitalize on a plethora of training data and feature extraction processes that are uniquely configured to recognize impairment indicators with higher accuracy than traditional methods, thus presenting a considerable technical advance.

Moreover, the technology is implemented through an innovative use of machine learning models that traverse through diverse data types, including video and audio, by adopting layers of data abstraction and synthesis. Such models are trained and iteratively improved using supervised learning, enhancing the learning algorithm's ability to dynamically adapt to new data and situations encountered in the field. By doing so, this computerized assessment tool exhibits an unprecedented level of real-time adaptability and precision in determining the likelihood of impairment.

The inventive system further addresses technical problems surrounding environmental and physiological noise reduction that arise during the administration of SFSTs. Through the application of noise filtering algorithms and normalization techniques within the motion and audio data processing pipeline, the system provides a robust data analysis framework. It minimizes false positives and negatives which are prevalent in manual SFST evaluations. This attribute is pivotal as it elevates the system's usability and reliability across varied and unpredictable field conditions. Thus, the system not only processes information more effectively but does so in a manner wherein the technical apparatus surpasses mere data collection and enters the realm of insightful, technologically enhanced decision support tools.

By offering a tangible technological solution to the subjective nature of traditional sobriety tests, these embodiments reflect a transformative approach that redefines field sobriety testing with precision-engineered enhancements deeply embedded in the realm of computer science and technology.

Any of the embodiments described herein can be performed with the help of one or more computers programmed with artificial intelligence, machine learning, machine vision, neural networks, or any combination of detecting, analysis, and presentation techniques. The system may employ local processors, cloud-based computer infrastructure or a combination.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The disclosure sets forth example embodiments and, as such, is not intended to limit the scope of embodiments of the disclosure and the appended claims in any way. Embodiments have been described above with the aid of functional building blocks illustrating the implementation of specified functions and relationships thereof. The boundaries of these functional building blocks have been arbitrarily defined herein for the convenience of the description. Alternate boundaries can be defined to the extent that the specified functions and relationships thereof are appropriately performed.

The foregoing description of specific embodiments will so fully reveal the general nature of embodiments of the disclosure that others can, by applying knowledge of those of ordinary skill in the art, readily modify and/or adapt for various applications such specific embodiments, without undue experimentation, without departing from the general concept of embodiments of the disclosure. Therefore, such adaptation and modifications are intended to be within the meaning and range of equivalents of the disclosed embodiments, based on the teaching and guidance presented herein. The phraseology or terminology herein is for the purpose of description and not of limitation, such that the terminology or phraseology of the specification is to be interpreted by persons of ordinary skill in the relevant art in light of the teachings and guidance presented herein.

The breadth and scope of embodiments of the disclosure should not be limited by any of the above-described example embodiments, but should be defined only in accordance with the following claims and their equivalents.

Conditional language, such as, among others, “can,” “could,” “might,” or “may,” unless specifically stated otherwise, or otherwise understood within the context as used, is generally intended to convey that certain implementations could include, while other implementations do not include, certain features, elements, and/or operations. Thus, such conditional language generally is not intended to imply that features, elements, and/or operations are in any way required for one or more implementations or that one or more implementations necessarily include logic for deciding, with or without user input or prompting, whether these features, elements, and/or operations are included or are to be performed in any particular implementation.

Unless otherwise noted, the terms “connected to” and “coupled to” (and their derivatives), as used in the specification, are to be construed as permitting both direct and indirect (i.e., via other elements or components) connection. In addition, the terms “a” or “an,” as used in the specification, are to be construed as meaning “at least one of” Finally, for ease of use, the terms “including” and “having” (and their derivatives), as used in the description do not preclude additional components and are to be construed as open ended.

The specification and annexed drawings disclose examples of systems, apparatus, devices, and techniques that may provide a system and method for determining acoustical signatures of discharged firearms. It is, of course, not possible to describe every conceivable combination of elements and/or methods for purposes of describing the various features of the disclosure, but those of ordinary skill in the art recognize that many further combinations and permutations of the disclosed features are possible. Accordingly, various modifications may be made to the disclosure without departing from the scope or spirit thereof. Further, other embodiments of the disclosure may be apparent from consideration of the specification and annexed drawings, and practice of disclosed embodiments as presented herein. Examples put forward in the specification and annexed drawings should be considered, in all respects, as illustrative and not restrictive. Although specific terms are employed herein, they are used in a generic and descriptive sense only, and not used for purposes of limitation.

Those skilled in the art will appreciate that, in some implementations, the functionality provided by the processes and systems discussed above may be provided in alternative ways, such as being split among more software programs or routines or consolidated into fewer programs or routines. Similarly, in some implementations, illustrated processes and systems may provide more or less functionality than is described, such as when other illustrated processes instead lack or include such functionality respectively, or when the amount of functionality that is provided is altered. In addition, while various operations may be illustrated as being performed in a particular manner (e.g., in serial or in parallel) and/or in a particular order, those skilled in the art will appreciate that in other implementations the operations may be performed in other orders and in other manners. Those skilled in the art will also appreciate that the data structures discussed above may be structured in different manners, such as by having a single data structure split into multiple data structures or by having multiple data structures consolidated into a single data structure. Similarly, in some implementations, illustrated data structures may store more or less information than is described, such as when other illustrated data structures instead lack or include such information respectively, or when the amount or types of information that is stored is altered. The various methods and systems as illustrated in the figures and described herein represent example implementations. The methods and systems may be implemented in software, hardware, or a combination thereof in other implementations. Similarly, the order of any method may be changed and various elements may be added, reordered, combined, omitted, modified, etc., in other implementations.

From the foregoing, it will be appreciated that, although specific implementations have been described herein for purposes of illustration, various modifications may be made without deviating from the spirit and scope of the appended claims and the elements recited therein. In addition, while certain aspects are presented below in certain claim forms, the inventors contemplate the various aspects in any available claim form. For example, while only some aspects may currently be recited as being embodied in a particular configuration, other aspects may likewise be so embodied. Various modifications and changes may be made as would be obvious to a person skilled in the art having the benefit of this disclosure. It is intended to embrace all such modifications and changes and, accordingly, the above description is to be regarded in an illustrative rather than a restrictive sense.

What I claim is:

1. A method for determining impairment, comprising:
instructing a subject to perform a standardized field sobriety test (SFST);
receiving, from a video capture device, video data of the subject;
determining one or more body landmarks of the subject;
generating a wireframe model of the subject, the wireframe model indicating the one or more body landmarks;
tracking, by a computing device executing a machine learning model, the one or more body landmarks during performance of the SFST to generate motion data associated with the one or more body landmarks;
generating an animation of the wireframe model of the subject based on motion of the one or more body landmarks during performance of the SFST;
determining, based on the motion data associated with the one or more body landmarks, a score associated with the performance of the SFST; and
generating a likelihood that the subject is impaired.

2. The method of claim 1, wherein receiving the video data includes capturing video data by a mobile phone.

3. The method of claim 1, further comprising receiving audio data from an audio capture device, the audio data associated with the subject.

4. The method of claim 3, further comprising determining an audio score associated with the audio data and wherein determining the score associated with the performance of the SFST is based, at least in part, on the audio score.

5. The method of claim 1, wherein tracking the one or more body landmarks includes generating a bounding box around each of the one or more body landmarks.

6. The method of claim 1, further comprising executing a machine learning model to correlate the motion data with the score.

7. The method of claim 1, wherein the one or more body landmarks include a pupil of an eye.

8. The method of claim 1, wherein the one or more body landmarks include a foot.

9. The method of claim 1, wherein the one or more body landmarks include a center of mass.

10. A method for determining impairment of a subject, comprising:
instructing the subject to perform one or more standard field sobriety tests (SFST);
receiving video data of a body motion of the subject performing the one or more SFST;
determining one or more body landmarks viewable in the video data of the body motion;
tracking the one or more body landmarks during an action;
generating, based at least in part on the tracking the one or more body landmarks, motion data;
determining a score associated with the motion data;
associating the motion data with the score; and
generating, based at least in part on the motion data and the score, a likelihood that the subject is impaired.

11. The method of claim 10, wherein receiving the video data includes capturing the video data by a mobile computing device.

12. The method of claim 10, further comprising executing a machine learning model to correlate the motion data with the score.

13. The method of claim 10, further comprising training a machine learning model on training data associated with performance of one or more SFSTs.

14. The method of claim 10, further comprising receiving an audible response and performing natural language processing on the audible response.

15. The method of claim 10, further comprising receiving motion data from a wearable sensor.

16. The method of claim 10, wherein instructing the subject is comprises presenting, through a speaker, audible instructions for performing the one or more SFST.

17. The method of claim 10 wherein generating a likelihood that the subject is impaired is performed by a machine learning model.

18. The method of claim 17, wherein the machine learning model is iteratively trained on training data through supervised learning.

19. A system for determining impairment of a subject through objective measures, comprising:
a data acquisition unit configured to capture video data and audio data of a subject performing a standardized field sobriety test (SFST), wherein the video data includes visual inputs of the subject's body movement and the audio data includes auditory inputs of the spoken instructions and responses;
a computing device operably coupled with the data acquisition unit, the computing device comprising:
a processor configured to execute a machine learning model that analyzes the video data to identify and track a plurality of body landmarks of the subject during the SFST to generate motion data, wherein the body landmarks include at least a pupil of an eye, a foot, and a center of mass;
a memory storing instructions which, when executed by the processor, cause the computing device to process the audio data using natural language processing to assess speech patterns;
a correlation module configured to integrate the motion data and assessed speech patterns to determine a score indicative of the subject's performance on the SFST;
a decision engine operatively configured to generate, based on the score, a likelihood that the subject is impaired using a supervised machine learning algorithm trained on historical SFST performance data; and
an output module configured to present the generated likelihood of impairment and associated analysis in a human-readable format.

20. The system of claim 19, wherein the data acquisition unit comprises a mobile computing device with an integrated camera and microphone, the camera positioned to capture a front-facing view of the subject, and the microphone configured to capture ambient noise and subject speech, wherein the captured data is utilized to enhance the accuracy of the motion data and speech pattern assessment by employing real-time noise reduction algorithms.
